(19) United States
(12) Patent Application Publication
(10) Pub. No.: US 2005/0187631 A1
(43) Pub. Date: Aug. 25, 2005

(54) Prosthetic device

(75) Inventors: James E. Van Hoeck, Cordova, TN (US); Carlos E. Gil, Collierville, TN (US); Randall N. Allard, Germantown, TN (US); Greg Marik, Germantown, TN (US); Hai H, Trieu, Cordova, TN (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 11/096,385

(22) Filed: Apr. 1, 2005

Related U.S. Application Data
(63) Continuation-in-part of application No. 10/830,247, filed on Apr. 22, 2004.
Application No. 10/830,247 is a continuation-in-part of application No. 10/765,260, filed on Jan. 27, 2004.

(51) Int. Cl. A61F 2/44

(52) U.S. Cl. 623/17.13; 623/17.14

(57) ABSTRACT

A prosthetic device according to which a time-dependent degree of resistance is provided to at least one predetermined type of relative directional motion between first and second components of the device.

### CROSS-REFERENCE TO RELATED APPLICATIONS

This application is a continuation-in-part of U.S. utility patent application Ser. No. 10/830,247, attorney docket no. 31132.194, filed on Apr. 22, 2004, which is a continuation-in-part of U.S. utility patent application Ser. No. 10/765,260, attorney docket No. 31132.173, filed on Jan. 27, 2004, the disclosures of which are incorporated by reference.

### BACKGROUND

The present disclosure relates generally to prosthetic devices and systems and in particular to prosthetic devices and systems that provide spinal stabilization.

Spinal discs that extend between the endplates of adjacent vertebrae in a spinal column of the human body provide critical support between the adjacent vertebrae. These discs can rupture, degenerate and/or protrude by injury, degradation, disease or the like to such a degree that the intervertebral space between adjacent vertebrae collapses as the disc loses at least a part of its support function, which can cause impingement of the nerve roots and severe pain. In some cases, surgical correction may be required.

Typically, the surgical correction includes the removal of the spinal disc from between the adjacent vertebrae, and, in order to preserve the intervertebral disc space for proper spinal-column function, a prosthetic device is sometimes inserted between the adjacent vertebrae. In this context, prosthetic devices may be referred to as intervertebral prosthetic joints, prosthetic implants, disc prostheses or artificial discs, among other labels.

While preserving the intervertebral disc space for proper spinal-column function, most prosthetic devices permit at least one of the adjacent vertebrae to undergo different types of motion relative to the other, including bending and rotation. Bending may occur in several directions: flexion or forward bending, extension or backward bending, left-side bending (bending towards the human's left side), right-side bending (bending towards the human's right side), or any combination thereof. Rotation may occur in different directions: left rotation, that is, rotating towards the human's left side with the spinal column serving generally as an imaginary axis of rotation; and right rotation, that is, rotating towards the human's right side with the spinal column again serving generally as an imaginary axis of rotation.

In addition to the aforementioned motion types, some prosthetic devices further permit relative translation between the adjacent vertebrae in the anterior-posterior (front-to-back), posterior-anterior (back-to-front), medial-lateral right (middle-to-right side), or medial-lateral left (middle-to-left side) directions, or any combination thereof. Also, some prosthetic devices may permit combinations of the aforementioned types of motion.

### SUMMARY OF THE INVENTION

According to one aspect of the present invention, a prosthetic device adapted to be inserted between first and second vertebrae is provided that includes a first component adapted to engage the first vertebra; a second component adapted to engage the second vertebra; and means connected to at least one of the first and second components for providing a time-dependent degree of resistance to at least one predetermined type of relative directional motion between the first and second components.

According to another aspect of the present invention, a prosthetic device adapted to be inserted between first and second vertebrae is provided that includes a first component adapted to engage the first vertebra; a second component adapted to engage the second vertebra; and at least one element connected to at least one of the first and second components; wherein the prosthetic device has a first configuration in which the at least one element provides a first degree of resistance to at least one predetermined type of relative directional motion between the first and second components; and a second configuration in which the at least one element provides a second degree of resistance to the at least one predetermined type of relative directional motion between the first and second components wherein the second degree of resistance is less than the first degree of resistance.

According to another aspect of the present invention, a prosthetic system is provided that includes a device adapted to be inserted in an intervertebral space between adjacent first and second vertebrae; and means connected to at least one of the first and second vertebrae for providing a degree of resistance to at least one predetermined type of relative directional motion between the first and second vertebrae wherein the degree of resistance decreases over time.

According to another aspect of the present invention, a method is provided that includes inserting a prosthetic device between first and second vertebrae and providing a time-dependent degree of resistance to at least one type of predetermined relative directional motion between the first and second vertebrae after the step of inserting, wherein the at least one predetermined type of relative directional motion between the first and second vertebrae is selected from the group consisting of flexion, extension, left-side bending, right-side bending, left rotation, right rotation, medial-lateral right translation, medial-lateral left translation, anterior-posterior translation and posterior-anterior translation.

### BRIEF DESCRIPTION OF THE DRAWINGS

FIG. 1a is a perspective view of a prosthetic device according to an embodiment of the present invention.

FIG. 1b is an anterior section view of the device of FIG. 1a taken along line 1b-1b.

FIGS. 1c, 1d and 1e are views similar to that of FIG. 1b but depicting other operational modes.

FIG. 2a is a perspective view of a prosthetic device according to another embodiment of the present invention.

FIGS. 2b, 2c and 2d are views similar to that of FIG. 2a but depicting other operational modes.

FIG. 3a is a perspective view of a prosthetic device according to another embodiment of the present invention.

FIG. 3b is an anterior section view of the device of FIG. 3a taken along line 3b-3b.

FIG. 3c is a view similar to that of FIG. 3b but depicting another operational mode.

FIG. 4a is a perspective view of a prosthetic device according to another embodiment of the present invention.

FIG. 4b is an anterior section view of the device of FIG. 4a taken along line 4b-4b.

FIGS. 5, 6, 7 and 8 are anterior section views of prosthetic devices according to alternative embodiments of the present invention.

FIGS. 9, 10, 11 and 12 are anterior section views of prosthetic systems according to alternative embodiments of the present invention.

### DETAILED DESCRIPTION

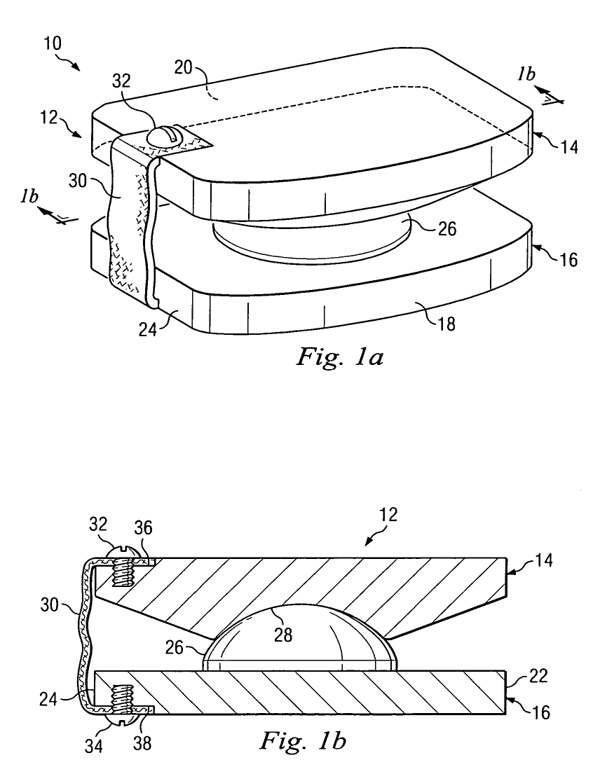

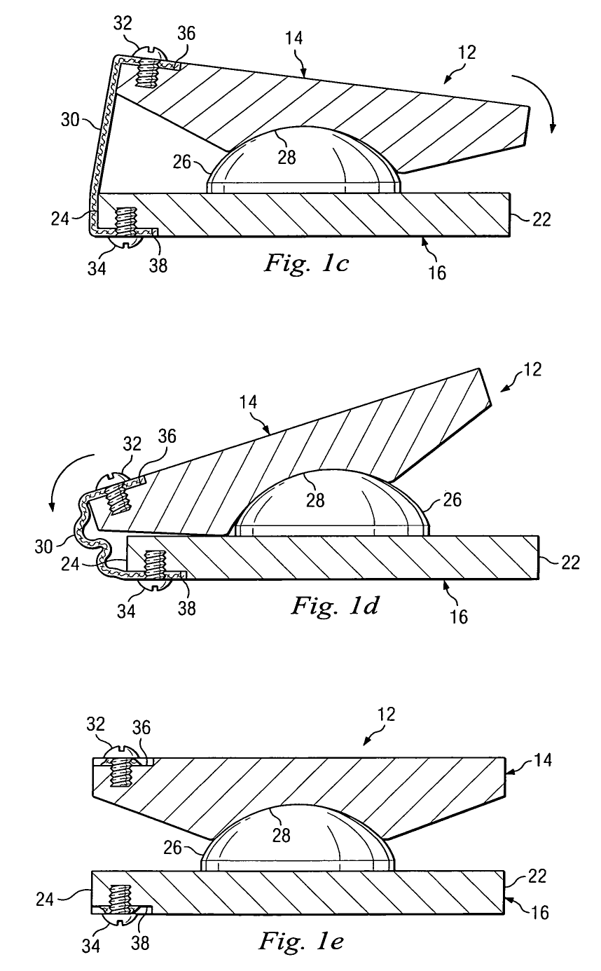

Referring to FIGS. 1a and 1b, a prosthetic device is generally referred to by the reference numeral 10. The device 10 includes a disc prosthesis or artificial disc 12 having a component in the form of an upper plate 14 and a component in the form of a lower plate 16. It is understood that the disc 12 is an articulating joint configured for insertion between adjacent vertebrae in a human spine so that the plates 14 and 16 each engage one of the adjacent vertebrae, and that the disc 12 maintains or restores motion by providing relative bending, translational and/or rotational motion between the adjacent vertebrae. The disc 12, along with the plates 14 and 16, includes an anterior side 18, a posterior side 20, a left lateral side 22 and a right lateral side 24. A convex-shaped projection 26 extends from the plate 16 and engages an articular surface defined by a concave recess 28 formed in the plate 14. The disc 12 may include additional structure and other features not shown but disclosed in detail in other patents and/or patent publications such as, for example, U.S. Pat. No. 6,740,118 to Eisermann et al., the disclosure of which is incorporated by reference.

An element in the form of a tether 30 extends between the plate 14 and the plate 16, and is composed of a material that is adapted to lose substance, degrade, decay or dissolve gradually over time. Suitable materials of which the tether 30 may be composed include, but are not limited to, any type of resorbable material, any type of bioresorbable material, any type of degradable material, any type of biodegradable material or any combination thereof. It is understood that suitable resorbable or bioresorbable materials, of which the tether 30 may be composed, may include, but are not limited to, polyactides, polyglycolides, tyrosine-derived polycarbonates, polyanhydrides, polyorthoesters, polyphosphazenes, bioactive glass, calcium phosphates such as hydroxyapatites, polycaprolactones, polydioxanones and combinations thereof, and various other polymer types, copolymer types and combinations thereof.

Threaded fasteners 32 and 34 connect the tether 30 to the plates 14 and 16, respectively. As shown in FIG. 1b, recesses 36 and 38 are formed in the plates 14 and 16, respectively, to receive the tether 30. The tether 30 is sized so that it is relaxed and not undergoing tension when the disc 12 is in its neutral position, as shown in FIG. 1b.

Prior to insertion of the device 10 between adjacent vertebrae in a spinal colunm, and immediately after insertion, the plates 14 and 16 may pivot and/or rotate relative to each other. In particular and referring to FIG. 1c, the device 10 may undergo left-side bending, that is, the plate 14 may pivot towards the left lateral side 22 of the plate 16 as shown by the arrow. After the plate 14 pivots to a certain predetermined degree, the tether 30 is tensioned so that it begins to resist or restrict this bending. As the plate 14 continues to pivot, the tether 30 continues to restrict or resist the left-side bending until the tether is tensioned to the point that it prevents any additional pivoting, as shown in FIG. 1c, thereby limiting the allowable range of left-side bending, and stabilizing the disc 12 and the spine engaged therewith.

Referring to FIG. 1d, the device 10 may undergo right-side bending, that is, the plate 14 may pivot towards the right lateral side 24 of the plate 16 as shown by the arrow. Unlike during left-side bending, the tether 30 provides no resistance or restriction, permitting the disc 12 to undergo maximum right-side bending, limited only by the physical design of the disc 12, including its plates 14 and 16.

The device 10 may undergo forward bending or flexion, wherein the plate 14 pivots towards the anterior side 18 of the plate 16, or backward bending or extension, wherein the plate 14 pivots towards the posterior side 20 of the plate 16. The tether 30 does not resist, limit or restrict this flexion or extension.

Also, the device 10 may undergo left rotation wherein the plate 14 rotates counterclockwise, or right rotation wherein the plate 14 rotates clockwise. The tether 30 is sized to resist or restrict left and right rotation of the plate 14 and ultimately to prevent predetermined excessive levels of left and right rotation of the plate 14, thereby limiting the allowable range of left and right rotation, and stabilizing the disc 12 and the spine engaged therewith. It is understood that, under certain conditions, the plate 16 may also experience the types of relative directional motion described above in connection with the plate 14, moving relative to the plate 14.

Due to the initial vertical extension of the tether 30, it is understood that left-side bending may be considered the primary motion restriction of interest whereas left and right rotation may be considered secondary motion restrictions of interest. Moreover, it is understood that the tether 30 may be sized so that it has no “slack” and therefore substantially prevents any left-side bending. Thus, it is understood that the degree of slack in the tether 30 at the neutral position controls the allowable range of motion in the selected direction, that is, the left-side bending range of motion.

In view of the foregoing, it is understood that, prior to the insertion of the device 10 between adjacent vertebrae in a spinal column, and immediately thereafter, the tether 30 provides an initial degree of resistance to at least three types of relative directional motion between the plates 14 and 16, that is, left-side bending, left rotation and right rotation.

However, after the device 10 has been inserted between the adjacent vertebrae, the material of the tether 30 gradually begins to lose substance, degrade, decay or dissolve within the human body. Thus, at any particular time after appreciable degradation of the tether 30 has begun, the degree of resistance provided by the tether 30 to left-side bending, left rotation and/or right rotation is less than the initial degree of resistance provided by the tether 30 immediately after the insertion of the device 10 between the adjacent vertebrae. Therefore, the tether 30 provides a time-dependent degree of resistance to left-side bending, left rotation and/or right rotation between the plates 14 and 16, with the degree of resistance decreasing over time. It is understood that, due to the engagement of the plates 14 and 16 with the respective ones of the adjacent vertebrae, the tether 30 provides a time-dependent degree of resistance to the foregoing predetermined types of relative directional motion between the adjacent vertebrae.

Referring to FIG. 1e, the tether 30 continues to lose substance, degrade, decay or dissolve until the device 10 is in a configuration in which the tether 30 is substantially nonexistent. As a result of the total or near-total loss of substance, degradation, decay or dissolution of the tether 30, the degree of resistance provided by the tether 30 is reduced to at least an insubstantial amount and full mobility to the disc 12 is restored, with the types of relative directional motion between the plates 14 and 16 being limited only by the physical design of the disc 12. That is, notwithstanding the total or near-total loss of substance, degradation, decay or dissolution of the tether 30, appreciable relative translation between the plates 14 and 16 is still not permitted, although flexion, extension, right-side bending, left-side bending and any combination thereof is permitted.

It is understood that human tissue and/or other natural structures in the vicinity of the device 10 may heal and grow back to their natural positions, occupying the space that was previously occupied by the tether 30. It is further understood that the time period over which the tether 30 completely or nearly completely loses substance, degrades, decays or dissolves may vary considerably and may be dependent upon several factors including, but not limited to, the specific properties of the material of the tether 30. For example, the time period for total or near-total degradation of the tether 30 may range from one to two years. Thus, the time-dependent degree of resistance provided by the tether 30 permits the human, into whose spinal column the device 10 has been inserted, to recuperate from the spinal surgery and slowly progress into a surgical or biomechanical equilibrium, without jeopardizing any desired initial stability that the disc 12 and the tether 30 are designed to provide.

Further, it is understood that, instead of the entire tether 30 gradually undergoing total or near-total loss of substance, degradation, decay or dissolution, only a portion of the tether 30 may gradually lose substance, degrade, decay or dissolve so that the tether 30 permanently continues to provide a degree of resistance to left-side bending, left rotation and/or right rotation. It is understood that any permanent degree of resistance provided by the tether 30 is less than the initial degree of resistance provided by the tether 30 immediately after the insertion of the device 10 between the adjacent vertebrae. In an exemplary embodiment, to effect a permanent degree of resistance, the tether 30 may be composed of a combination of resorbable and nonresorbable materials. Suitable nonresorbable materials include, but are not limited to, derived materials or any other solid materials, non-resorbable polymers, metal or any combination thereof. Polymer types may include, but are not limited to, polyethylene, polyester, polyaryletherketone, polyamide, polytetrafluoroethylene, polyurethane, silicone, copolymers of silicone and polyurethane with or without end-group modifications, hydrogels, polyolefin-based rubber, polyisobutylene, polyisoprene, neoprene, nitrile rubber and vulcanized rubber. Metal types may include, but are not limited to, stainless steel, titanium, titanium alloys, shape-memory alloys or any combination thereof.

Moreover, it is understood that different types of motion may be controlled in the device 10, that is, the tether 30 may be connected to the plates 14 and 16 on the anterior side 18, the posterior side 20 or the left lateral side 22 in order to primarily restrict extension, flexion or right-side bending, respectively (while also continuing to restrict left and right rotation).

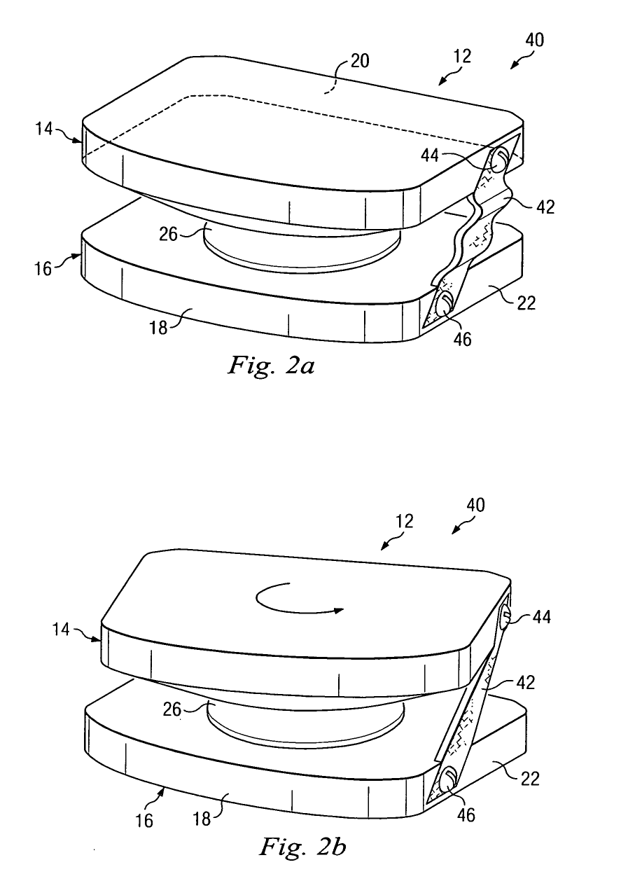

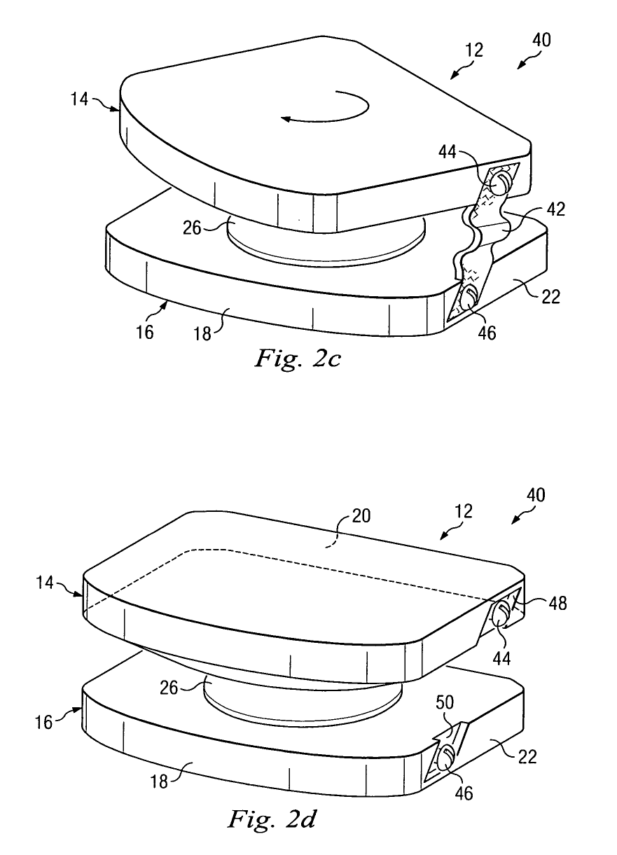

FIGS. 2a through 12 depict prosthetic devices and/or systems according to alternative embodiments of the present invention. It is understood that all of the artificial discs in these embodiments are configured for insertion between adjacent vertebrae in a human spine, and maintain or restore motion by providing relative directional motion between the vertebrae, as discussed above. Also, the terms “flexion,” “extension,” “left-side bending,” “right-side bending,” “left rotation,” “right rotation,” “medial-lateral right translation,” “medial-lateral left translation,” “anterior-posterior translation” and “posterior-anterior translation,” as discussed above, are applicable to one or more of the below-described embodiments.

Referring to FIG. 2a, another embodiment of a prosthetic device is generally referred to by the reference numeral 40, and is similar to that of FIGS. 1a through 1e and contains several parts of the embodiment which are given the same reference numerals. In the embodiment of FIGS. 2a and 2b, an element in the form of a tether 42 diagonally extends from the plate 14 to the plate 16 on the left lateral side 22. The tether 42 is composed of a material that is adapted to lose substance, degrade, decay or dissolve gradually over time. Suitable materials of which the tether 42 may be composed include, but are not limited to, those types of materials identified above in connection with the tether 30 of the embodiment of FIGS. 1a through 1e.

With continuing reference to FIG. 2a, threaded fasteners 44 and 46 connect the tether 42 to the plates 14 and 16, respectively, with the fastener 44 positioned towards the posterior side 20 and the fastener 46 positioned towards the anterior side 18. Recesses 48 and 50 (shown in FIG. 2d) are formed in the plates 14 and 16, respectively, to receive the ends of the tether 42. The tether 42 is sized so that it is relaxed and not undergoing tension when the disc 12 is in its neutral position, as shown in FIG. 2a.

Prior to insertion of the device 40 between adjacent vertebrae in a spinal column, and immediately after insertion, the plates 14 and 16 may pivot and/or rotate relative to each other. In particular and referring to FIG. 2b, the device 40 may undergo left rotation (or counterclockwise rotation). As shown by the arrow, the plate 14 rotates counterclockwise relative to the plate 16, and the tether 42 is tensioned so that it begins to restrict or resist the rotation. As the plate 14 continues to rotate, the tether 42 continues to restrict or resist the rotation until the tether substantially prevents any additional rotation, thereby stabilizing the disc 12 and the spine engaged therewith.

Referring to FIG. 2c, the device 40 may undergo right rotation as shown by the arrow. Unlike during left rotation, the tether 42 provides no resistance, permitting the disc 12 to undergo maximum right rotation, limited only by the physical design of the disc 12, including its plates 14 and 16. Similarly, the tether 42 does not restrict or resist flexion, extension, left-side bending or right-side bending.

Due to the initial diagonal extension of the tether 42, it is understood that left rotation is the primary motion restriction of interest for this embodiment. Moreover, it is understood that the tether 42 may be sized so that it substantially prevents any left rotation.

In view of the foregoing, it is understood that, prior to the insertion of the device 40 between adjacent vertebrae in a spinal column, and immediately thereafter, the tether 42 provides an initial degree of resistance to at least one type of relative directional motion between the plates 14 and 16, that is, left rotation.

However, after the device 40 has been inserted between the adjacent vertebrae, the material of the tether 42 gradually begins to lose substance, degrade, decay or dissolve within the human body. Thus, at any particular time after appreciable degradation of the tether 42 has begun, the degree of resistance provided by the tether 42 to left rotation is less than the initial degree of resistance provided by the tether 42 immediately after the insertion of the device 40 between the adjacent vertebrae. Therefore, the tether 42 provides a time-dependent degree of resistance to left rotation, with the degree of resistance decreasing over time.

Referring to FIG. 2d, the tether 42 continues to lose substance, degrade, decay or dissolve until the device 40 is in a configuration in which the tether 42 is substantially nonexistent. As a result of the total or near-total loss of substance, degradation, decay or dissolution of the tether 42, the degree of resistance provided by the tether 42 decreases to at least an insubstantial amount and full mobility to the disc 12 is restored, with the types of relative directional motion between the plates 14 and 16 being limited only by the physical design of the disc 12. That is, notwithstanding the total or near-total loss of substance, degradation, decay or dissolution of the tether 42, appreciable relative translation between the plates 14 and 16 is still not permitted, although flexion, extension, right-side bending, left-side bending, left rotation, right rotation and any combination thereof is permitted.

It is understood that human tissue and/or other natural structures in the vicinity of the device 40 may heal and grow back to their natural positions, occupying the space that was previously occupied by the tether 42. It is further understood that the time period over which the tether 42 completely or nearly completely loses substance, degrades, decays or dissolves may vary considerably and may be dependent upon several factors including, but not limited to, the specific properties of the material of the tether 42. For example, the time period for total or near-total degradation of the tether 42 may range from one to two years. Thus, the time-dependent degree of resistance provided by the tether 42 permits the human, into whose spinal column the device 40 has been inserted, to recuperate from the spinal surgery and slowly progress into a surgical or biomechanical equilibrium, without jeopardizing any desired initial stability that the disc 12 and the tether 42 are designed to provide.

Further, it is understood that, instead of the entire tether 42 gradually undergoing total or near-total loss of substance, degradation, decay or dissolution, only a portion of the tether 42 may gradually lose substance, degrade, decay or dissolve so that the tether 42 permanently continues to provide a degree of resistance to left-side bending, left rotation and/or right rotation. It is understood that any permanent degree of resistance provided by the tether 42 is less than the initial degree of resistance provided by the tether immediately after the insertion of the device between the adjacent vertebrae. In an exemplary embodiment, to effect a permanent degree of resistance, the tether 42 may be composed of a combination of resorbable and nonresorbable materials, with the nonresorbable materials including those types of materials identified above in connection with the tether 30 of the embodiment of FIGS. 1a through 1e.

Moreover, it is understood that the tether 42 may extend diagonally in a manner with the fastener 44 positioned towards the anterior side 18 and the fastener 46 positioned towards the posterior side 20, so that right rotation is restricted. Still further, it is understood that the tether 42 may extend from the plate 14 to the plate 16 on the anterior side 18, the posterior side 20 or the left lateral side 22.

Also, for the embodiments of FIGS. 1a through 1e, and FIGS. 2a through 2d, it is understood that the type of artificial disc 12 and the quantity, designs, arrangements and/or attachment techniques of the tethers 30 and 42 may vary considerably. Examples of such variations are disclosed in U.S. utility patent application Ser. No. 10/830,247, attorney docket no. 31132.194, filed on Apr. 22, 2004, which is a continuation-in-part of U.S. utility patent application Ser. No. 10/765,260, attorney docket no. 31132.173, filed on Jan. 27, 2004, the disclosures of which are incorporated by reference. Thus, it is understood that the quantity and/or combinations of the predetermined types of relative directional motion between the plates 14 and 16, to which the tethers 30 and/or 42 provide degrees of resistance, may vary considerably.

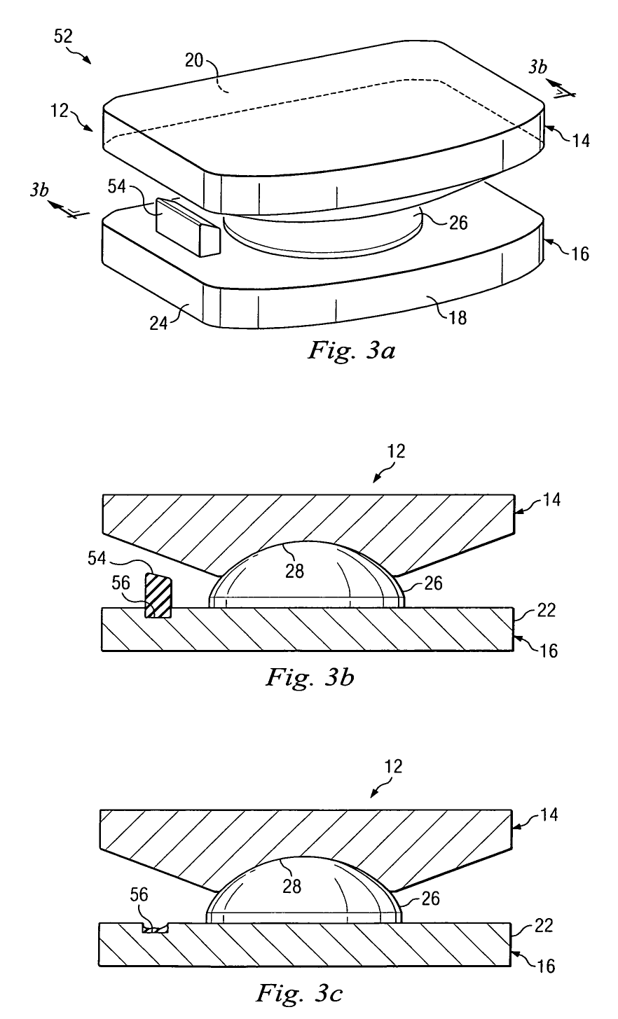

Referring to FIGS. 3a and 3b, another embodiment of a prosthetic device is generally referred to by the reference numeral 52, and is similar to that of FIGS. 1a through 1e and contains parts of the embodiment which are given the same reference numerals. In the embodiment of FIGS. 3a and 3b, an element in the form of a bumper 54 is disposed in a channel 56 formed in the plate 16 and extends towards the plate 14. The bumper 54 is composed of a material that is adapted to at least partially lose substance, degrade, decay or dissolve gradually over time. Suitable materials of which the bumper 54 may be composed include, but are not limited to, all of the types of materials identified above in connection with the tether 30 of the embodiment of FIGS. 1a through 1e, and any combination thereof.

The operation of the prosthetic device 52 is similar to that of the operation of the prosthetic devices 10 and 40 in the embodiments of FIGS. 1a through 1e and FIGS. 2a through 2d, respectively, and therefore will not be described in detail. Prior to the insertion of the device 52 between adjacent vertebrae in a spinal column, and immediately thereafter, the bumper 54 provides an initial degree of resistance to at least one type of relative directional motion between the plates 14 and 16, that is, right-side bending. In particular, as the plate 14 undergoes right-side bending and pivots towards the right lateral side 24 of the plate 16, the plate 14 eventually contacts the bumper 54, and the bumper 54 resists and/or prevents any further right-side bending of the plate 14.

After insertion of the device 52 between the adjacent vertebrae, and as shown in FIG. 3c, the bumper 54 gradually loses substance, degrades, decays or dissolves over time until the device 52 is in a configuration in which the bumper 54 is substantially nonexistent. As a result of the total or near-total loss of substance, degradation, decay or dissolution of the bumper 54, the degree of resistance provided by the bumper 54 decreases to at least an insubstantial amount and full mobility to the disc 12 is restored, with the types of relative directional motion between the plates 14 and 16 being limited only by the physical design of the disc 12.

Therefore, in a manner similar to the tethers 30 and 42 described above in connection with the embodiments of FIGS. 1a through 1e and FIGS. 2a through 2d, respectively, the bumper 54 provides a time-dependent degree of resistance to at least right-side bending, with the degree of resistance decreasing over time. It is understood that the location and/or size of the bumper 54 may be varied, and/or additional bumpers may be added, in order to provide a degree of resistance to one or more other predetermined relative directional motion types between the plates 14 and 16.

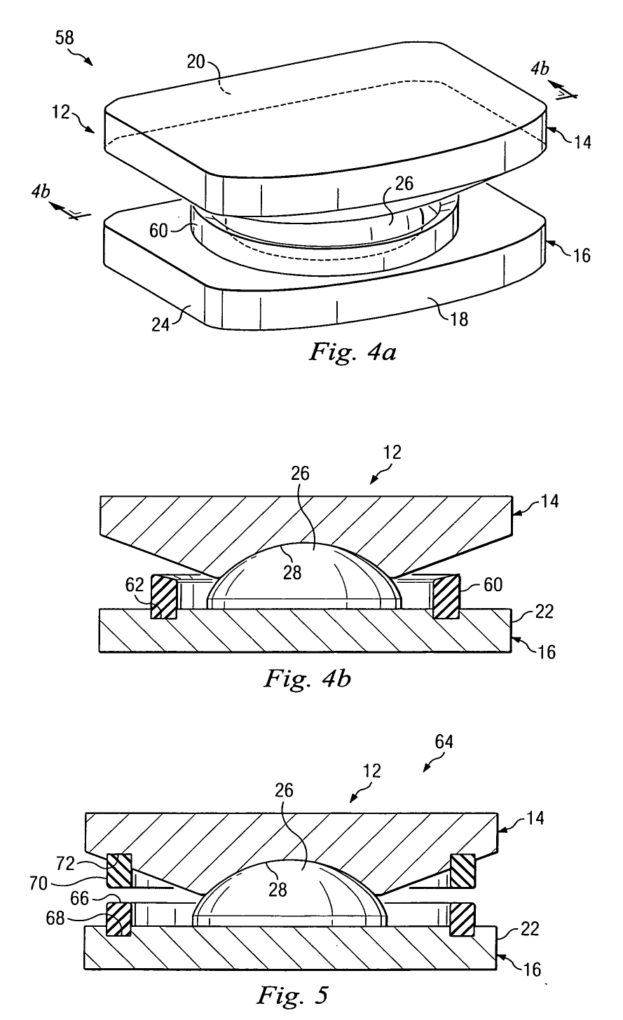

Referring to FIGS. 4a and 4b, another embodiment of a prosthetic device is generally referred to by the reference numeral 58, and is similar to that of FIGS. 1a through 1e and contains parts of the embodiment which are given the same reference numerals. In the embodiment of FIGS. 4a and 4b, an element in the form of a ring 60 is disposed in a channel 62 formed in the plate 16, extending towards the plate 14 and circumferentially extending about the projection 26. The ring 60 is composed of a material that is adapted to at least partially lose substance, degrade, decay or dissolve gradually over time. Suitable materials of which the ring 60 may be composed include, but are not limited to, all of the types of materials identified above in connection with the tether 30 of the embodiment of FIGS. 1a through 1e, and any combination thereof.

The operation of the prosthetic device 58 is similar to that of the operation of the prosthetic devices 10 and 40 in the embodiments of FIGS. 1a through 1e and FIGS. 2a through 2d, respectively, and therefore will not be described in detail. Prior to the insertion of the device 58 between adjacent vertebrae in a spinal column, and immediately thereafter, the ring 60 provides an initial degree of resistance to several types of relative directional motion between the plates 14 and 16, including left-side bending, right-side bending, flexion and extension.

Thereafter, in a manner similar to the tethers 30 and 42 described above in connection with the embodiments of FIGS. 1a through 1e and FIGS. 2a through 2d, respectively, the ring 60 provides a time-dependent degree of resistance to left-side bending, right-side bending, flexion and extension, with the degree of resistance decreasing over time.

Referring to FIG. 5, another embodiment of a prosthetic device is generally referred to by the reference numeral 64, and is similar to that of FIGS. 4a and 4b and contains parts of the embodiment which are given the same reference numerals. In the embodiment of FIG. 5, an element in the form of a ring 66 is disposed in a channel 68 formed in the plate 16, extending towards the plate 14 and circumferentially extending about the projection 26. An element in the form of a ring 70 is disposed in a channel 72 formed in the plate 14, extending towards the plate 16 and circumferentially extending about the recess 28.

The rings 66 and 70 are composed of materials that are adapted to at least partially lose substance, degrade, decay or dissolve gradually over time. Suitable materials of which the rings 66 and 70 may be composed include, but are not limited to, all of the types of materials identified above in connection with the tether 30 of the embodiment of FIGS. 1a through 1e, and any combination thereof.

The operation of the prosthetic device 58 is similar to that of the operation of the prosthetic device 58 in the embodiment of FIGS. 4a and 4b and therefore will not be described in detail.

It is understood that the rings 66 and 70 provide an initial degree of resistance to several types of relative directional motion between the plates 14 and 16, including left-side bending, right-side bending, flexion and extension. It is further understood that, due to the proximity between the distal end portions of the rings 66 and 70, the allowable ranges of motion for the device 64 are less than the allowable ranges of motion for the device 58 in the embodiment of FIGS. 4a and 4b. That is, when the device 64 undergoes, for example, left-side bending, the plate 14 pivots to the left lateral side 22 of the plate 16 until the distal end of the ring 70 contacts the distal end portion of the ring 66 so that contact between the rings 66 and 70 resist and/or prevent further left-bending, resulting in a smaller range of left-bending motion than the range of left-bending motion permitted for the device 58 in the embodiment of FIGS. 4a and 4b, in which the plate 14 contacts the bumper 60 to resist and/or prevent further left-bending. Thus, in the embodiment of FIG. 5, the device 64 initially maintains a more neutral position than the device 58 in the embodiment of FIGS. 4a and 4b.

In addition to the above-described disc 12, it is understood that the prosthetic devices described above may be comprised of all types of disc prostheses or artificial discs, including articulating, non-articulating, elastic articulating, elastic or flexible disc designs. Although the disc 12 is an example of an articulating disc, other types of articulating discs known to those skilled in the art may be used.

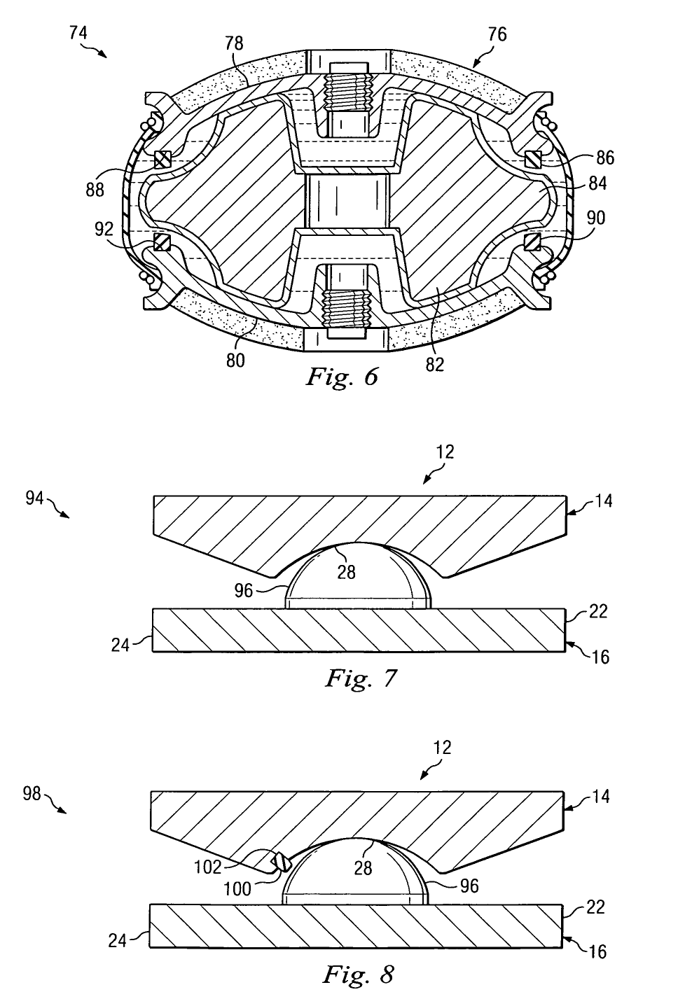

For example, referring to FIG. 6, another embodiment of a prosthetic device is generally referred to by the reference numeral 74. An artificial disc 76 includes a pair of components in the form of shells 78 and 80 and a component in the form of a central body 82 disposed between the shells 78 and 80, the central body 82 having an equatorial ridge 84. An element in the form of a ring 86 is disposed in a channel 88 and at least partially circumferentially extends about the central body 82. Similarly, an element in the form of a ring 90 is disposed in a channel 92 and at least partially circumferentially extends about the central body 82. The additional structure and other features of the artificial disc 76 are disclosed in U.S. Patent Publication Nos. 2002/0128715 (Ser. No. 09/924,298) and 2003/0135277 (Ser. No. 10/303,569), the disclosures of which are incorporated by reference.

The rings 86 and 90 are composed of materials that are adapted to at least partially lose substance, degrade, decay or dissolve gradually over time. Suitable materials of which the rings 66 and 70 may be composed include, but are not limited to, all of the types of materials identified above in connection with the tether 30 of the embodiment of FIGS. 1a through 1e, and any combination thereof.

The disc 76 is adapted to permit several types of relative directional motion between the shells 78 and 80 including left-bending, right-bending, flexion, extension, anterior-posterior translation, posterior-anterior translation, medial-lateral left translation and medial-lateral right translation ranges of motion. Moreover, the disc 76 is adapted to permit anterior-posterior translation, posterior-anterior translation, medial-lateral left translation and medial-lateral right translation between either one of the shells 78 and 80 and the other of the shells 78 or 80 and/or the central body 82.

The operation of the prosthetic device 74 is similar to that of the operation of the prosthetic devices 10 and 40 in the embodiments of FIGS. 1a through 1e and FIGS. 2a through 2d, respectively, and therefore will not be described in detail. It is understood that, prior to the insertion of the device 74 between adjacent vertebrae in a spinal column, and immediately thereafter, the rings 86 and 90 provide an initial degree of resistance to several types of relative directional motion between the shells 78 and 80, including left-side bending, right-side bending, flexion and extension. For example, when the disc 76 undergoes right-side bending, the shell 78 may pivot or bend counterclockwise, as viewed in FIG. 6, until the ring 86 contacts the equatorial ridge 84 of the central body 82 and/or until the equatorial ridge 84 contacts the ring 90, resisting and/or preventing further left-bending motion.

It is further understood that, prior to the insertion of the device 74 between adjacent vertebrae in a spinal column, and immediately thereafter, the rings 86 and 90 provide an initial degree of resistance to several types of relative directional motion between one of the shells 78 and 80 and the other of the shells 78 and 80 and/or the central body 82, including anterior-posterior translation, posterior-anterior translation, medial-lateral left translation and medial-lateral right translation. For example, the shell 78 may undergo medial-lateral left translation, that is, the shell 78 may move to the right as viewed in FIG. 6, until contact occurs between the ring 86 and the central body 82 and/or contact occurs between the ring 90 and/or the central body 82.

Thereafter, in a manner similar to the tethers 30 and 42 described above in connection with the embodiments of FIGS. 1a through 1e and FIGS. 2a through 2d, respectively, the rings 86 and 90 provide a time-dependent degree of resistance to left-side bending, right-side bending, flexion, extension, anterior-posterior translation, posterior-anterior translation, medial-lateral left translation and medial-lateral right translation with the degree of resistance decreasing over time.

Referring to FIG. 7, another embodiment of a prosthetic device is generally referred to by the reference numeral 94, and is similar to that of FIGS. 1a through 1e and contains several parts of the embodiment which are given the same reference numerals. In the embodiment of FIG. 7, a convex-shaped projection 96 extends from the plate 16 and engages a surface defined by the concave recess 28 formed in the plate 14. The projection 96 is sized so that only a portion of the surface defined by the projection 96 engages the surface defined by the recess 28.

In operation, both before and after the prosthetic device 94 is inserted between adjacent vertebrae in a spinal column, the prosthetic device 94 permits relative directional motion between the plates 14 and 16 in several directions including flexion, extension, left-side bending, right-side bending, left rotation, right rotation, anterior-posterior translation, posterior-anterior translation, medial-lateral left translation, medial-lateral right translation and/or any combination thereof.

It is understood that appreciable anterior-posterior translation, posterior-anterior translation, medial-lateral left translation or medial-lateral right translation between the plates 14 and 16 is permitted because the dimensions of the projection 96 and the recess 28 are such that one or more gaps exist between one or more corresponding portions of the surfaces defined by the projection 96 and the recess 28, thereby permitting these types of relative directional motion.

It is further understood that a hardenable fluidic material that is adapted to at least partially lose substance, degrade, decay or dissolve gradually over time, such as, for example, resorbable cement, may be applied to and/or between one or more of the surfaces defined by the projection 96 and the recess 28 so that, prior to the insertion of the device 94 between adjacent vertebrae in a spinal column, and immediately thereafter, one or more types of relative directional motion between the plates 14 and 16 is prevented including flexion, extension, left-side bending, right-side bending, left rotation, right rotation, anterior-posterior translation, posterior-anterior translation, medial-lateral left translation, medial-lateral right translation and/or any combination thereof.

Thereafter, in a manner similar to the tethers 30 and 42 described above in connection with the embodiments of FIGS. 1a through 1e and FIGS. 2a through 2d, respectively, the resorbable cement provides a time-dependent degree of resistance to flexion, extension, left-side bending, right-side bending, left rotation, right rotation, anterior-posterior translation, posterior-anterior translation, medial-lateral left translation, medial-lateral right translation and/or any combination thereof, with the degree of resistance decreasing over time.

It is further understood that the projection 96 and/or the recess 28 may be sized so that the great majority of the surface defined by the projection 96 contacts the great majority of the surface defined by the recess 28, thereby resisting and/or preventing appreciable anterior-posterior translation, posterior-anterior translation, medial-lateral left translation and/or medial-lateral right translation between the plates 14 and 16, regardless of whether a hardenable fluidic material is applied to and/or disposed between the projection 96 and the recess 28.

It is further understood that the sizes, shapes, cross-sections and/or dimensions of the projection 96 and the recess 28, and may be varied considerably to restrict and/or prevent a wide variety of predetermined types of relative directional motion between the plates 14 and 16, regardless of whether a hardenable fluidic material is applied to and/or disposed between the projection 96 and the recess 28.

Referring to FIG. 8, another embodiment of a prosthetic device is generally referred to by the reference numeral 98, and is similar to that of FIG. 7 and contains several parts of the embodiment which are given the same reference numerals. In the embodiment of FIG. 8, an element in the form of a bumper 100 is disposed in a channel 102 formed in the surface defined by the recess 28. The bumper 100 is composed of a material that is adapted to at least partially lose substance, degrade, decay or dissolve gradually over time. Suitable materials of which the bumper 100 may be composed include, but are not limited to, all of the types of materials identified above in connection with the tether 30 of the embodiment of FIGS. 1a through 1e, and any combination thereof.

The operation of the prosthetic device 98 is similar to that of the prosthetic device 94 of the embodiment of FIG. 7, except that prior to the insertion of the device 98 between adjacent vertebrae in a spinal column, and immediately thereafter, the bumper 100 provides an initial degree of resistance to one or more types of relative directional motion between the plates 14 and 16, including medial-lateral left translation and right-side bending. In particular, the plate 14 may undergo medial-lateral left translation and the plate 14 may move to the right, as viewed in FIG. 6, until contact occurs between the bumper 100 and the projection 96, and further medial-lateral left translation is thereby resisted and/or prevented. Moreover, the plate 14 may undergo right-side bending and the plate 14 may pivot or rotate counterclockwise, as viewed in FIG. 6, until contact occurs between the bumper 100 and the projection 96, and further right-side bending is thereby resisted and/or prevented.

Thereafter, in a manner similar to the tethers 30 and 42 described above in connection with the embodiments of FIGS. 1a through 1e and FIGS. 2a through 2d, respectively, the bumper 100 provides a time-dependent degree of resistance to right-side bending and medial-lateral left translation, with the degree of resistance decreasing over time.

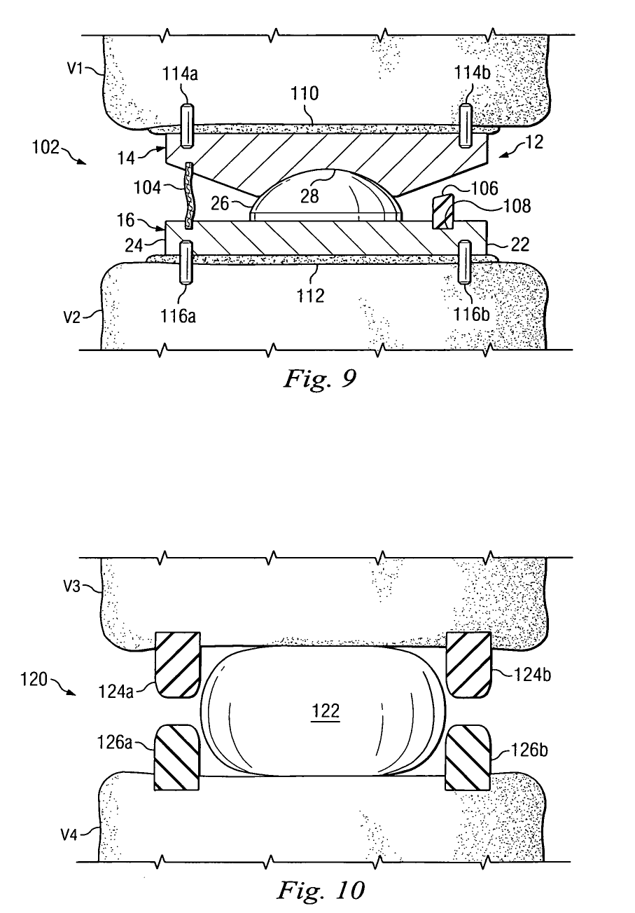

Referring to FIG. 9, another embodiment of a prosthetic device is generally referred to by the reference numeral 102 and is shown inserted between adjacent vertebrae V1 and V2. The embodiment of FIG. 9 is similar to that of FIGS. 1a through 1e and contains several parts of the embodiment which are given the same reference numerals. In the embodiment of FIG. 9, the plates 14 and 16 are shown engaged with the vertebrae V1 and V2, respectively.

An element in the form of a tether 104 is connected to and extends between the plates 14 and 16. An element in the form of a bumper 106 is disposed in a channel 108 formed in the plate 16 and extends towards the plate 14. The tether 104 and the bumper 106 are each composed of a material that is adapted to at least partially lose substance, degrade, decay or dissolve gradually over time. Suitable materials of which the tether 104 and the bumper 106 may each be composed include all of the types of materials identified above in connection with the tether 30 of the embodiment of FIGS. 1a through 1e, and any combination thereof.

Layers or mantles of a hardenable fluidic material 110 and 112, that are adapted to at least partially lose substance, degrade, decay or dissolve gradually over time, such as, for example, mantles of resorbable cement, are disposed between the plate 14 and the vertebra V1, and between the plate 16 and the vertebra V2, respectively. Resorbable anchors 114a and 114b extend from the plate 14 and into the vertebra V1. Likewise, resorbable anchors 116a and 116b extend from the plate 16 and into the vertebra V2.

In operation, and immediately after the insertion of the device 102 between the vertebrae V1 and V2, as shown in FIG. 9, the resorbable anchors 114a and 114b serve to fix or anchor the plate 14 to the vertebra V1. Moreover, the mantle of material 110 serves to fix the engagement between the plate 14 and the vertebra V1, and to fill in any voids or openings formed in the surfaces of the plate 14 and the vertebra V1 to improve the engagement therebetween. Thus, any relative movement between the device 102 and the vertebra V1 is resisted and/or prevented.

Likewise, the resorbable anchors 116a and 116b serve to fix or anchor the plate 16 to the vertebra V2. Moreover, the mantle of material 112 serves to fix the engagement between the plate 16 and the vertebra V2, and to fill in any voids or openings formed in the surfaces of the plate 16 and the vertebra V2 to improve the engagement therebetween. Thus, any relative movement between the device 102 and the vertebra V2 is resisted and/or prevented.

The tether 104 provides an initial degree of resistance to at least three types of relative directional motion between the plates 14 and 16, that is, left-side bending, left rotation and right rotation. The bumper 108 provides an initial degree of resistance to at least one type of relative directional motion between the plates 14 and 16, that is, left-side bending. It is understood that, due to the engagement of the plates 14 and 16 with the vertebrae V1 and V2, respectively, it follows that the tether 104 and the bumper 108 also provide initial degrees of resistance to the foregoing predetermined types of relative directional motion between the vertebrae V1 and V2, respectively.

Over a period of time after the device 102 has been inserted between the vertebrae V1 and V2, the anchors 114a, 114b, 116a and 116b begin to gradually resorb as osseous integration occurs at the interfaces between the anchors 114a and 114b and the vertebra V1, and between the anchors 116a and 116b and the vertebra V2, and bone begins to grow and occupy the spaces previously occupied by the anchors 114a, 114b, 116a and 116b. Thus, over time, the fixation strength provided by the anchors 114a, 114b, 116a and 116b decreases as the fixation strength provided by bone growth increases. Therefore, at any particular time after one or more of the anchors 114a, 114b, 116a and 116b have begun to resorb, the degree of resistance provided by one or more of the anchors 114a, 114b, 116a and 116b to relative movement between the plate 14 and the vertebra V1, and/or between the plate 16 and the vertebra V2, is less than the initial degree of resistance provided by the anchors 114a, 114b, 116a and 116b immediately after the insertion of the device 102 between the adjacent vertebrae V1 and V2.

Similarly, the mantles of material 110 and 112 also begin to gradually lose substance, degrade, decay or dissolve and bone begins to grow at the interface between the device 102 and the vertebrae V1 and V2, and the new bone growth begins to occupy the spaces previously occupied by the mantles of material 110 and 112. Thus, over time, the fixation strength provided by the mantles of material 110 and 112 decreases as the fixation strength provided by the bone growth increases. Therefore, at any particular time after the mantles of material 110 and 112 have begun to resorb, the degree of resistance provided by the mantles of material 110 and 112 to relative movement between the plate 14 and the vertebra V1, and between the plate 16 and the vertebra V2, is less than the initial degree of resistance provided by the mantles of material 110 and 112 immediately after the insertion of the device 102 between the adjacent vertebrae V1 and V2.

The tether 104 also begins to gradually lose substance, degrade, decay or dissolve within the human body. Thus, at any particular time after appreciable degradation of the tether 104 has begun, the degree of resistance provided by the tether 104 to left-side bending, left rotation and/or right rotation is less than the initial degree of resistance provided by the tether 104 immediately after the insertion of the device 102 between the vertebrae V1 and V2. Therefore, the tether 104 provides a time-dependent degree of resistance to left-side bending, left rotation and/or right rotation, with the degree of resistance decreasing over time.

The tether 104 continues to lose substance, degrade, decay or dissolve until it is substantially nonexistent. As a result of the total or near-total loss of substance, degradation, decay or dissolution of the tether 104, the degree of resistance provided by the tether 104 is reduced to at least an insubstantial amount.

Similarly, the bumper 108 also gradually loses substance, degrades, decays or dissolves over time until it is substantially nonexistent. As a result of the total or near-total loss of substance, degradation, decay or dissolution of the bumper 54, the degree of resistance provided by the bumper 54 decreases to at least an insubstantial amount. Therefore, the bumper 108 provides a time-dependent degree of resistance to at least right-side bending, with the degree of resistance decreasing over time.

After the degrees of resistance provided by the tether 104 and the bumper 108 have decreased to insubstantial amounts, full mobility to the disc 12 is restored, with the types of relative directional motion between the plates 14 and 16 being limited only by the physical design of the disc 12.

It is understood that the tether 104 and/or the bumper 108 may be sized, and/or the materials of the tether 104 and/or the bumper 108 may be selected, in order to provide a consistent rate of decreasing resistance over time so that the tether 104 and the bumper 108 become substantially nonexistent at approximately the same time.

It is further understood that the tether 104 and/or the bumper 108 may be sized, and/or the materials of the tether 104 and/or the bumper 108 may be selected, so that the tether 104 becomes substantially nonexistent before the bumper 108, or vice versa, lengthening the duration of motion resistance and/or prevention due to the tether 104 and/or the bumper 108.

It is further understood that one or more of the mantles of material 110 and 112 may be removed from the system 102.

Referring to FIG. 10, an embodiment of a prosthetic system is generally referred to by the reference numeral 120 and is shown inserted between adjacent vertebrae V3 and V4. A prosthetic device 122 in the form of a nuclear or nucleus replacement device engages the vertebrae V3 and V4. It is understood that the device 122 may be generally oval shaped, or may be generally spherically shaped, and/or may be generally spirally wound. It is further understood that the device 122 may be in the form of a Fernstrom Ball, or may be in the form of any type of nuclear or nucleus replacement device known to those generally skilled in the art, or any variation thereof.

Bumpers 124a and 124b are embedded in the endplate of the vertebra V3 and extend downwards into the intervertebral space between the vertebrae V3 and V4, and to the left and right, respectively, of the device 122, as viewed in FIG. 10. Bumpers 126a and 126b are embedded in the endplate of the vertebra V4 and extend upwards into the intervertebral space between the vertebrae V3 and V4, and to the left and right, respectively, of the device 122, as viewed in FIG. 10. It is understood that, instead of or in addition to embedment, the bumpers 124a, 124b, 126a and 126b may be connected to the vertebrae V3 and V4 using other techniques such as, for example, with fasteners.

The bumpers 124a, 124b, 126a and 126b are each composed of a material that is adapted to at least partially lose substance, degrade, decay or dissolve gradually over time. Suitable materials of which the bumpers 124a, 124b, 126a and 126b may each be composed include all of the types of materials identified above in connection with the tether 30 of the embodiment of FIGS. 1a through 1e, and any combination thereof.

In operation, and immediately after the implementation of the system 120 between the vertebrae V3 and V4, the bumpers 124a and 126a resist and/or prevent the device 122 from undergoing medial-lateral right translation, and the bumpers 124b and 126b resist and/or prevent the device 122 from undergoing medial-lateral left translation.

If the vertebra V3 begins to undergo medial-lateral left translation relative to the vertebra V4, contact between the bumper 124a and the device 122, and/or between the device 122 and the bumper 126b, resists and/or prevents the medial-lateral left translation. In a similar manner, if the vertebra V4 begins to undergo medial-lateral left translation relative to the vertebra V3, contact between the bumper 126a and the device 122, and/or between the device 122 and the bumper 124b, resists and/or prevents the medial-lateral left translation.

If the vertebra V3 begins to undergo medial-lateral right translation relative to the vertebra V4, contact between the bumper 124b and the device 122, and/or between the device 122 and the bumper 126a, resists and/or prevents the medial-lateral right translation. In a similar manner, if the vertebra V4 begins to undergo medial-lateral right translation relative to the vertebra V3, contact between the bumper 126b and the device 122, and/or between the device 122 and the bumper 124a, resists and/or prevents the medial-lateral right translation.

If the vertebra V3 undergoes left-side bending, contact between the distal end portions of the bumpers 124b and 126b resist and/or prevent the left-side bending. If the vertebra V3 undergoes right-side bending, contact between the distal end portions of the bumpers 124a and 126a resist and/or prevent the right-side bending. It is understood that, under certain conditions, the vertebra V4 may also experience the types of relative directional motion described above in connection with the vertebra V3, moving relative to the vertebra V3.

In view of the foregoing, it is understood that the bumpers 124a, 124b, 126a and 126b initially contain the device 122 within the intervertebral space between the vertebrae V3 and V4, at least with respect to the medial-lateral left and medial-lateral right translation directions. It is further understood that additional bumpers may added to the system 120, with the additional bumpers extending on the anterior and posterior sides of the device 122. In such an arrangement, the device 122 initially may be entirely contained within bumpers that resisting and/or prevent any type of translation of the device 122, the vertebra V3 and/or the vertebra V4 in any direction, and any type of bending of the vertebra V3 or V4, including flexion, extension, left-side bending and right-side bending.

Over a period of time after the implementation of the system 120 between the vertebrae V3 and V4, the bumpers 124a, 124b, 126a and 126b gradually lose substance, degrade, decay or dissolve over time until they are substantially nonexistent. As a result of the total or near-total loss of substance, degradation, decay or dissolution of the bumpers 124a, 124b, 126a and 126b, the degrees of resistance provided by the bumpers 124a, 124b, 126a and 126b decrease to at least an insubstantial amount. Thereafter, full mobility between the vertebrae V3 and V4 is permitted, as long as the device 122 remains in place between the vertebrae V3 and V4. Therefore, in a manner similar to the tethers 30 and 42 described above in connection with the embodiments of FIGS. 1a through 1e and FIGS. 2a through 2d, respectively, the bumpers 124a, 124b, 126a and 126b provide time-dependent degrees of resistance.

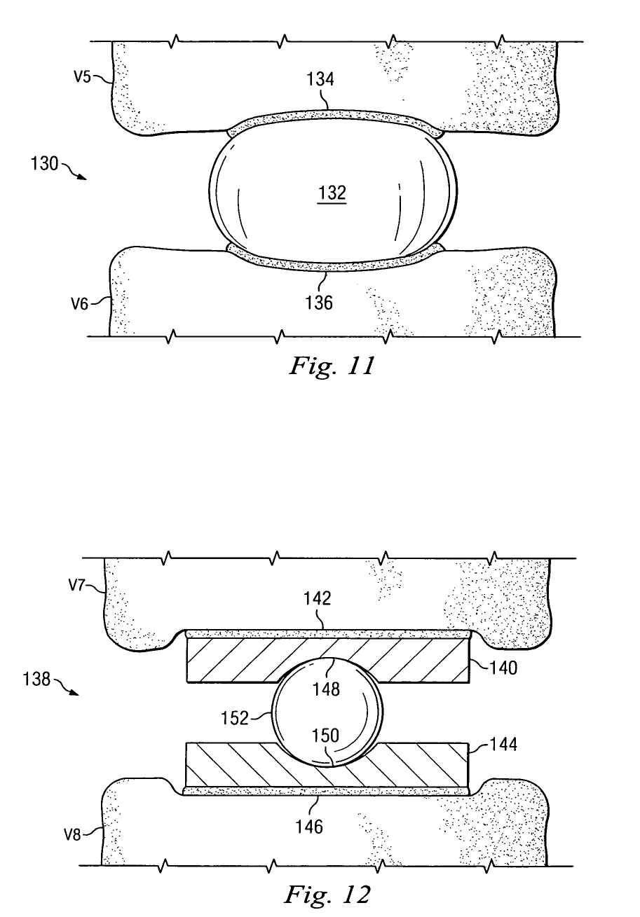

Referring to FIG. 11, another embodiment of a prosthetic system is generally referred to by the reference numeral 130 and is shown inserted between adjacent vertebrae V5 and V6. A prosthetic device 132 in the form of an articular bean engages the vertebrae V5 and V6. It is understood that the device 132 may be generally oval shaped, or may be generally spherically shaped. It is further understood that the device 132 may be in the form of a Fernstrom Ball, or may be in the form of any type of known nuclear device or nucleus replacement device, or any variation thereof.

Layers of hardenable fluidic material 134 and 136 are disposed between the device 132 and the vertebra V5, and between the device 132 and the vertebra V6, respectively. The layers of hardenable fluidic material 134 and 136 are composed of a material that is adapted to at least partially lose substance, degrade, decay or dissolve gradually over time, such as, for example, resorbable cement.

In operation, and immediately after the implementation of the system 130 between the vertebrae V5 and V6, the layers of hardenable fluidic material 134 and 136 resist and/or prevent one or more types of relative directional motion between the vertebrae V5 and V6, including flexion, extension, left-side bending, right-side bending, left rotation, right rotation, anterior-posterior translation, posterior-anterior translation, medial-lateral left translation, medial-lateral right translation and/or any combination thereof. Moreover, the layers of hardenable fluidic material 134 and 136 resist and/or prevent relative movement in any direction between the device 132 and the vertebrae V5 and/or V6. Thus, the system 130 initially maintains the vertebrae 134 and 136, and the intervertebral space therebetween, in substantially neutral positions.

Over a period of time after the implementation of the system 130 between the vertebrae V5 and V6, the layers of hardenable fluidic material 134 and 136 gradually lose substance, degrade, decay or dissolve over time until they are substantially nonexistent. As a result of the total or near-total loss of substance, degradation, decay or dissolution of the layers of hardenable fluidic material 134 and 136, the degrees of resistance provided the layers of hardenable fluidic material 134 and 136 decrease to at least an insubstantial amount. Thereafter, full mobility between the vertebrae V5 and V6 is permitted, as long as the device 132 remains in place between the vertebrae V5 and V6. Therefore, in a manner similar to the tethers 30 and 42 described above in connection with the embodiments of FIGS. 1a through 1e and FIGS. 2a through 2d, respectively, the layers of hardenable fluidic material 134 and 136 provide time-dependent degrees of resistance.

It is understood that one of the layers of fluidic material 134 and 136 may be removed from the system 130, thereby initially allowing motion of one of the vertebrae V5 or V6 relative to the other of the vertebrae V5 or V6 and the device 132. If one of the layers of fluidic material 134 and 136 are so removed, the above-described time-dependent resistance provided by the remaining layer may result in greater ranges of motion between vertebrae V5 and V6. It is further understood that bone ingrowth at the interfaces between the device 132 and the vertebrae V5 and V6 may be promoted in any conventional manner such as by, for example, applying a porous coating to the external surface of the device 136.

Referring to FIG. 12, another embodiment of a prosthetic system is generally referred to by the reference numeral 138 and is shown inserted between adjacent vertebrae V7 and V8. A component in the form of a plate 140 is engaged with the vertebra V7 and a layer of hardenable fluidic material 142 is disposed between the plate 140 and the vertebra V7. Similarly, a component in the form of a plate 144 is engaged with the vertebra V8 and a layer of hardenable fluidic material 146 is disposed between the plate 144 and the vertebra V8. The layers of hardenable fluidic material 142 and 146 are adapted to at least partially lose substance, degrade, decay or dissolve gradually over time, and may be in the form of, for example, layers of resorbable cement.

Concave-shaped recesses 148 and 150 are formed in the plates 140 and 144, respectively. A component in the form of a ball 152 is disposed between the plates 140 and 144 and contacts the surfaces defined by the recesses 148 and 150.

In operation, and immediately after implementation of the system 138 between the vertebrae V7 and V8, the layer of hardenable fluidic material 142 serves to fix the engagement between the plate 140 and the vertebra V7, and to fill in any voids or openings formed in the surfaces of the plate 140 and the vertebra V7 to improve the engagement therebetween. Thus, any relative movement between the plate 140 and the vertebra V7 is resisted and/or prevented by the layer of hardenable fluidic material 142.

Likewise, the layer of hardenable fluidic material 146 serves to fix the engagement between the plate 144 and the vertebra V8, and to fill in any voids or openings formed in the surfaces of the plate 144 and the vertebra V8 to improve the engagement therebetween. Thus, any relative movement between the plate 144 and the vertebra V8 is resisted and/or prevented by the layer of hardenable fluidic material 146.

Due to the contact between the ball 152 and the surface defined by the recess 148, the plate 140 and the vertebra V7 are able to undergo several types of directional motion relative to the plate 144 and the vertebra V8, including flexion, extension, left-side bending, right-side bending, left rotation and right rotation.

Over a period of time after the system 138 has been implemented between the vertebrae V7 and V8, the layers of hardenable fluidic material 142 and 146 begin to gradually lose substance, degrade, decay or dissolve and bone begins to grow at the interfaces between the plate 140 and the vertebra V7, and between the plate 144 and the vertebra V8. This new bone growth begins to occupy the spaces previously occupied by the layers of hardenable fluidic material 142 and 146. Thus, over time, the fixation strength provided by the layers of hardenable fluidic material 142 and 146 decreases as the fixation strength provided by the bone growth increases, and this decrease may result in greater ranges of motion between the vertebrae V7 and V8.

Therefore, at any particular time after the layers of hardenable fluidic material 142 and 146 have begun to resorb, the degrees of resistance provided by the layers of hardenable fluidic material 142 and 146 to relative movement between the plate 140 and the vertebra V7, and between the plate 144 and the vertebra V8, respectively, are less than the initial degrees of resistance provided by the layers of hardenable fluidic material 142 and 146 immediately after the implementation of the system 138 between the adjacent vertebrae V7 and V8. It is understood that the plate 140 and/or 144 may be backed with a polymetal compound. It is further understood that the plate 140 and the layer of hardenable fluidic material 142 may be removed from the system 138 so that the ball 152 directly engages the vertebra V7. Similarly, it is understood that the plate 144 and the layer of hardenable fluidic material 146 may be removed from the system 138 so that the ball 152 directly engages the vertebra V8.

A prosthetic device adapted to be inserted between first and second vertebrae has been described that includes a first component adapted to engage the first vertebra; a second component adapted to engage the second vertebra; and means connected to at least one of the first and second components for providing a time-dependent degree of resistance to at least one predetermined type of relative directional motion between the first and second components.

A prosthetic device adapted to be inserted between first and second vertebrae has been described that includes a first component adapted to engage the first vertebra; a second component adapted to engage the second vertebra; and at least one element connected to at least one of the first and second components; wherein the prosthetic device has a first configuration in which the at least one element provides a first degree of resistance to at least one predetermined type of relative directional motion between the first and second components; and a second configuration in which the at least one element provides a second degree of resistance to the at least one predetermined type of relative directional motion between the first and second components wherein the second degree of resistance is less than the first degree of resistance.

A prosthetic system has been described that includes a device adapted to be inserted in an intervertebral space between adjacent first and second vertebrae; and means connected to at least one of the first and second vertebrae for providing a degree of resistance to at least one predetermined type of relative directional motion between the first and second vertebrae wherein the degree of resistance decreases over time.

A method has been described that includes inserting a prosthetic device between first and second vertebrae and providing a time-dependent degree of resistance to at least one type of predetermined relative directional motion between the first and second vertebrae after the step of inserting, wherein the at least one predetermined type of relative directional motion between the first and second vertebrae is selected from the group consisting of flexion, extension, left-side bending, right-side bending, left rotation, right rotation, medial-lateral right translation, medial-lateral left translation, anterior-posterior translation and posterior-anterior translation.

It is understood that any foregoing spatial references, such as “upper,” “lower,” “above,” “below,” “between,” “vertical,” “angular,” “up,” “down,” “right,” “left,” etc., are for the purpose of illustration only and do not limit the specific orientation or location of the structure described above.

Moreover, it is understood that the quantities, shapes, sizes and cross-sections of the above-described tethers, bumpers and/or rings may be varied. It is further understood that additional elements in the form of bumpers, rings, tethers and/or variations thereof, and/or combinations and/or multiple series thereof, may be added to one or more of the above-described embodiments to provide time-dependent degrees of resistance to a wide variety of motion types or combinations thereof. It is further understood that each of the above-described tethers, bumpers and/or rings may have varying magnitudes of resistance to the predetermined types of relative directional motion between adjacent vertebrae and/or the components engaged therewith.

Also, it is understood that each of the above-described embodiments may be combined in whole or in part with one or more of the other above-described embodiments. It is further understood that each of the above-described embodiments may be combined in whole or in part with other components, devices, systems, methods and/or surgical techniques known to those skilled in the art to provide spinal stabilization and/or motion preservation.

Although exemplary embodiments of this invention have been described in detail above, those skilled in the art will readily appreciate that many other modifications are possible in the exemplary embodiments without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

1. A prosthetic device adapted to be inserted between first and second vertebrae, the device comprising a first component adapted to engage the first vertebra; a second component adapted to engage the second vertebra; and means connected to at least one of the first and second components for providing a time-dependent degree of resistance to at least one predetermined type of relative directional motion between the first and second components.

2. The device of claim 1 wherein the at least one predetermined type of relative directional motion between the first and second components is selected from the group consisting of flexion, extension, left-side bending, right-side bending, left rotation, right rotation, medial-lateral right translation, medial-lateral left translation, anterior-posterior translation and posterior-anterior translation.

3. The device of claim 2 wherein the time-dependent degree of resistance to the at least one predetermined type of relative directional motion between the first and second components decreases over time.

4. The device of claim 3 wherein the means comprises at least one element connected to the at least one of the first and second components.

5. The device of claim 4 wherein the at least one element is composed of a material that is adapted to at least partially lose substance gradually over time after the device is inserted between the first and second vertebrae.

6. The device of claim 5 wherein the at least one element is in the form of a ring extending from the at least one of the first and second components.

7. The device of claim 5 wherein the at least one element is in the form of a bumper extending from the at least one of the first and second components.

8. The device of claim 5 wherein the at least one element is in the form of a tether extending between the first and second components.

9. The device of claim 5 wherein the element permits at least one other predetermined type of relative directional motion between the first and second components.

10. The device of claim 9 wherein the at least one other predetermined type of relative directional motion between the first and second components is opposite in direction to the at least one predetermined type of relative directional motion between the first and second components.

11. The device of claim 9 wherein the at least one other predetermined type of relative directional motion between the first and second components is selected from the group consisting of flexion, extension, left-side bending, right-side bending, left rotation, right rotation, medial-lateral right translation, medial-lateral left translation, anterior-posterior translation and posterior-anterior translation.

12. The device of claim 3 further comprising a third component disposed between the first and second components; wherein the means comprises a pair of elements extending from respective ones of the first and second components; and wherein the elements provide a time-dependent degree of resistance to at least one predetermined type of relative directional motion between the third component and the at least one of the first and second components.

13. The device of claim 3 wherein the means comprises a layer of hardenable fluidic material that is adapted to at least partially lose substance gradually over time after the device is inserted between the first and second vertebrae.

14. The device of claim 3 wherein the first component comprises a recess formed therein; and wherein the second component comprises a projection extending therefrom so that at least a portion of the projection engages at least a portion of a surface defined by the recess.

15. The device of claim 14 wherein the means comprises at least one element wherein the at least one element is composed of a material that is adapted to at least partially lose substance gradually over time after the device is inserted between the first and second vertebrae.

16. A prosthetic device adapted to be inserted between first and second vertebrae, the device comprising:
a first component adapted to engage the first vertebra;
a second component adapted to engage the second vertebra; and
at least one element connected to at least one of the first and second components;
wherein the prosthetic device has:
a first configuration in which the at least one element provides a first degree of resistance to at least one predetermined type of relative directional motion between the first and second components; and
a second configuration in which the at least one element provides a second degree of resistance to the at least one predetermined type of relative directional motion between the first and second components wherein the second degree of resistance is less than the first degree of resistance.

17. The device of claim 16 wherein the at least one predetermined type of relative directional motion between the first and second components is selected from the group consisting of flexion, extension, left-side bending, right-side bending, left rotation, right rotation, medial-lateral right translation, medial-lateral left translation, anterior-posterior translation and posterior-anterior translation.

18. The device of claim 17 wherein the at least one element is composed of a material that is adapted to at least partially lose substance gradually over time after the device is inserted between the first and second vertebrae.

19. The device of claim 18 wherein the at least one element is selected from the group consisting of a tether, a bumper and a ring.

20. A prosthetic system comprising a device adapted to be inserted in an intervertebral space between adjacent first and second vertebrae; and means connected to at least one of the first and second vertebrae for providing a degree of resistance to at least one predetermined type of relative directional motion between the first and second vertebrae wherein the degree of resistance decreases over time.

21. The system of claim 20 wherein the at least one predetermined type of relative directional motion between the first and second vertebrae is selected from the group consisting of flexion, extension, left-side bending, right-side bending, left rotation, right rotation, medial-lateral right translation, medial-lateral left translation, anterior-posterior translation and posterior-anterior translation.

22. The system of claim 21 wherein the means comprises a layer of resorbable cement disposed between the device and the at least one of the first and second vertebrae.

23. The system of claim 22 wherein the device comprises an articular bean wherein the layer of resorbable cement is disposed between the articular bean and the at least one of the first and second vertebrae.

24. The system of claim 23 wherein the means further comprises another layer of resorbable cement disposed between the articular bean and at least one other of the first and second vertebrae.

25. The system of claim 22 wherein the device comprises a plate engaged with the at least one of the first and second vertebrae wherein the layer of resorbable cement is disposed between the plate and the at least one of the first and second vertebrae.

26. The system of claim 21 wherein the means comprises at least one element extending from the at least one of the first and second vertebrae.

27. The system of claim 26 wherein the device is in the form of a nuclear device; and wherein at least one element is in a form selected from the group consisting of a bumper and a ring.

28. A method comprising inserting a prosthetic device between first and second vertebrae and providing a time-dependent degree of resistance to at least one type of predetermined relative directional motion between the first and second vertebrae after the step of inserting, wherein the at least one predetermined type of relative directional motion between the first and second vertebrae is selected from the group consisting of flexion, extension, left-side bending, right-side bending, left rotation, right rotation, medial-lateral right translation, medial-lateral left translation, anterior-posterior translation and posterior-anterior translation.

29. The method of claim 28 wherein the step of providing comprises disposing a layer of resorbable cement between the device and at least one of the first and second vertebrae.

30. The method of claim 28 wherein the step of inserting comprises engaging a first component of the device with the first vertebra and engaging a second component of the device with the second vertebra; and wherein the step of providing comprises connecting at least one element to at least one of the first and second components wherein the at least one element is composed of a material that is adapted to at least partially lose substance gradually over time after the step of inserting.
